(19) United States
(12) Patent Application Publication
(10) Pub. No.: US 2011/0315146 A1
(43) Pub. Date: Dec. 29, 2011

(54) INFANT NASAL CPAP CANULA SEAL DEFINING A VENT

(76) Inventors: Timothy R. Beevers, McMinnville, OR (US); Katherine K. Beevers, McMinnville, OR (US)

(21) Appl. No.: 12/825,017

(22) Filed: Jun. 28, 2010

(51) Int. Cl. A61M 16/06

(52) U.S. Cl. 128/207.13

(57) ABSTRACT

A nasal CPAP canula seal having directions of “above” and “below” by its anticipated placement on an infant face, above being higher up on the face and below being lower down. The seal has a canula supporting portion, defining holes for canula passage into the nostrils, and a nose-anchoring portion extending about the apertures and designed to help anchor the nasal CPAP canula seal by adhering to the infant nose. Also, the nose-anchoring portion defines a laterally central notch cut-out adapted to form a moisture vent when the nose-anchoring portion is folded about the nose.

### BACKGROUND

Continuous positive airway pressure is a method of sustaining the breathing of fragile infants (typically prematurely born infants) by gently blowing air into the nostrils to maintain positive pressure in the airways, thereby keeping the airways open. Canula seals that gently adhere to the infants nose, cheek and upper part of the upper lip, and that provide a pair of apertures for supporting the canulas that are placed into the infant nostrils to deliver the air have been commercially available. Also, canula seals have been made by neo-natal nurses by cutting a pattern from wound dressing. Those using these canula seals have noticed some phenomena that have not been 100% desirable. Sometimes a portion of the canula seal would peel away from the infant's face. Also, health care personnel would sometimes place some of the portion intended to cover the philtrum over the vermillion of the infant's upper lip.

In a different environment, these issues might have escaped notice entirely, but when dealing with premature infants, even the smallest issue draws attention. Accordingly, careful study has identified the source of these problems and to recognition of the desirability of design improvements.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In a first separate aspect, the present invention may take the form of a nasal CPAP canula seal having directions of “above” and “below” by its anticipated placement on an infant face, above being higher up on the face and below being lower down. The seal has a canula supporting portion, defining holes for canula passage into the nostrils, and a nose-anchoring portion extending about the apertures and designed to help anchor the nasal CPAP canula seal by adhering to the infant nose. Also, the nose-anchoring portion defines a laterally central notch cut-out adapted to form a moisture vent when the nose-anchoring portion is folded about the nose.

In a second separate aspect, the present invention may take the form of a method of applying a nasal CPAP canula seal in such a manner that a vent is provided to permit moisture to escape from the nose skin. The method utilizes a nasal CPAP canula seal that has a canula supporting portion, defining apertures for canula passage into the nostrils and a nose-anchoring portion, attached to the canula supporting portion and defining a notch. The method includes placing the nasal CPAP canula seal about the infant nose so that the apertures are coincident with the infant's nostrils and the notch is substantially over the tip of the infant nose, thereby forming a vent that permits moisture to escape.

In a third separate aspect, the present invention may take the form of a nasal CPAP canula seal having directions of “above” and “below” by its anticipated placement on an infant face, above being higher up on the face and below being lower down and thereby defining latitude as a set of lines going horizontally across the canula seal. The seal includes a canula supporting portion, defining apertures, in turn defining aperture centers, for canula passage into the nostrils, and a nose-anchoring portion extending about the apertures and designed to help anchor the nasal CPAP canula seal by adhering to the infant nose, the nose anchoring portion having at least one latitude of greatest width. Also, the aperture centers are within two millimeters of the latitude of greatest width.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced drawings. It is intended that the embodiments and figures disclosed herein are to be considered illustrative rather than restrictive.

FIG. 1 is a plan view of an infant nasal CPAP canula seal according to the present invention.

FIG. 2 is a perspective view of the seal of FIG. 1 in place on an infant's face.

FIG. 3 is a perspective view of the seal of FIG. 1 in place on an infant's face, supporting a CPAP canula.

### DETAILED DESCRIPTION OF THE PREFERRED EMBODIMENT

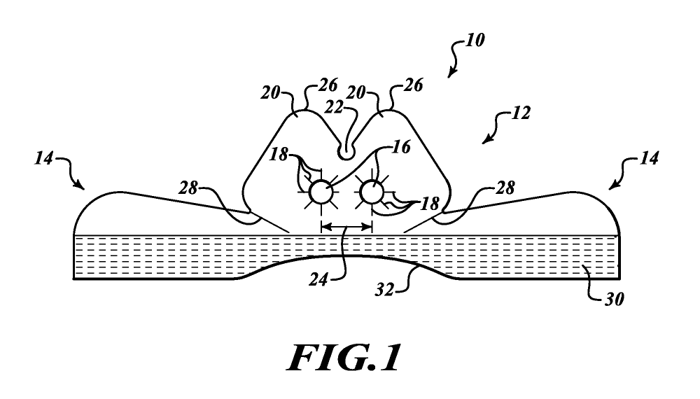

Referring to FIG. 1, a preferred embodiment of an infant nasal CPAP canula seal 10. Seal 10 is die-cut from a sheet of wound dressing material made of hydrocolloid laminated to a 25 micrometer thick sheet of polyurethane. The hydrocolloid gently adheres to the infant face. Seal 10 is sold in a clean package, and on a sheet of backing material, from which it is removed, prior to use.

In alternative preferred embodiments, the layer of seal 10 that contacts the infant face is made of silicone or acrylic adhesive. For example, Silbione 4512 A/B, which may be obtained from a distributor named on the website www.bluestarsilicones.com may be used. One advantage of this type of material is that it can be washed and yet still maintain its adherent properties. Skilled persons will recognize that the sheet of polyurethane may be replaced with any one of a broad range of biocompatible films that are widely available and many of which are clear. If both materials used are transparent, the seal 10 has an additional advantage, that the infant skin tissue will be more clearly visible underneath seal 10. This will help medical personnel quickly spot potential problems.

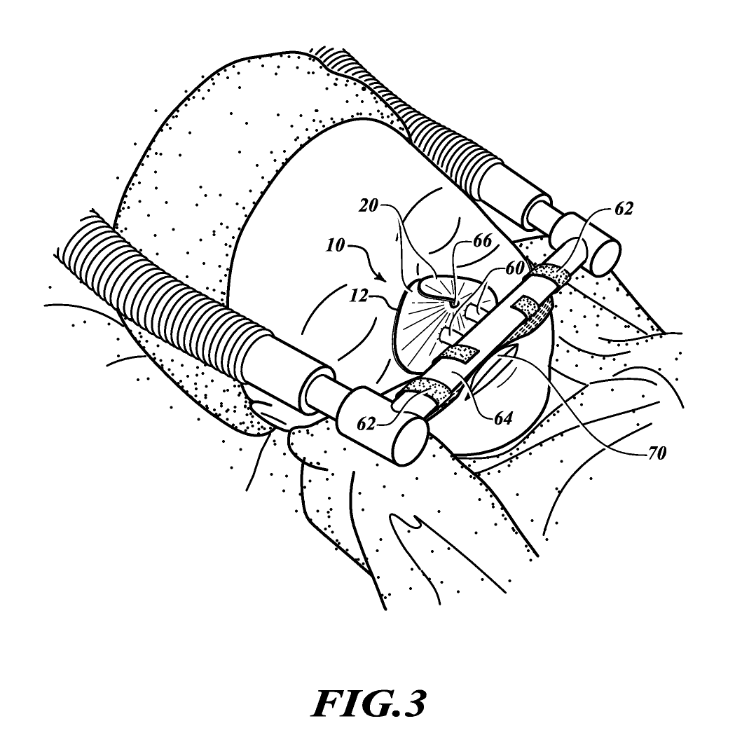

Seal 10 includes a nose covering portion 12 and a lip and cheek adhering portion 14. Nose covering portion 12 defines a pair of nostril apertures 16 for supporting the CPAP tubes 60 (FIG. 3) as they enter the nostrils. Radially emanating cuts or stellations 18, permit the resultant flaps to separate and protect the nostril interiors from direct contact with rigid tubes 60.

The nose covering portion 12 includes two petals 20 and defines a notch 22. In the use of prior art canula seals, it was found that during application of the seal, petals would sometime approach the eye of the infant. After analysis and experimentation, it was found that shorter rounded petals still provided sufficient adhesion and yet avoided this problem.

Apertures 16 define a center-to-center spacing length 24. It has been found that, across the various sizes seals 10, that if the length of petals 20 is kept below two center-to-center spacing lengths 24, that the danger of approaching the eye is minimized.

The acute angles of prior art seals appear to have provided a point for seal 10 to begin to separate from the skin to which it was adhered. It has been found that one way to avoid this problem is to avoid acute angles at the tips 26 of petals 20, in favor, ideally of an arcuate upper edge at tips 26. The definition of nose covering portion 12 is completed by a pair of cuts 28, permitting portion 12 to separate from portion 14 during application.

The bottom portion of lip and cheek adhering portion 14 is covered with hook material tape 30, that is applied during manufacturing and has outwardly projecting hooks. Referring to FIG. 3, loop material 62, wrapped about horizontal tube 64 sticks to this hook material 30, to keep the horizontal tube 64 and therefore the nose tubes 60 in place.

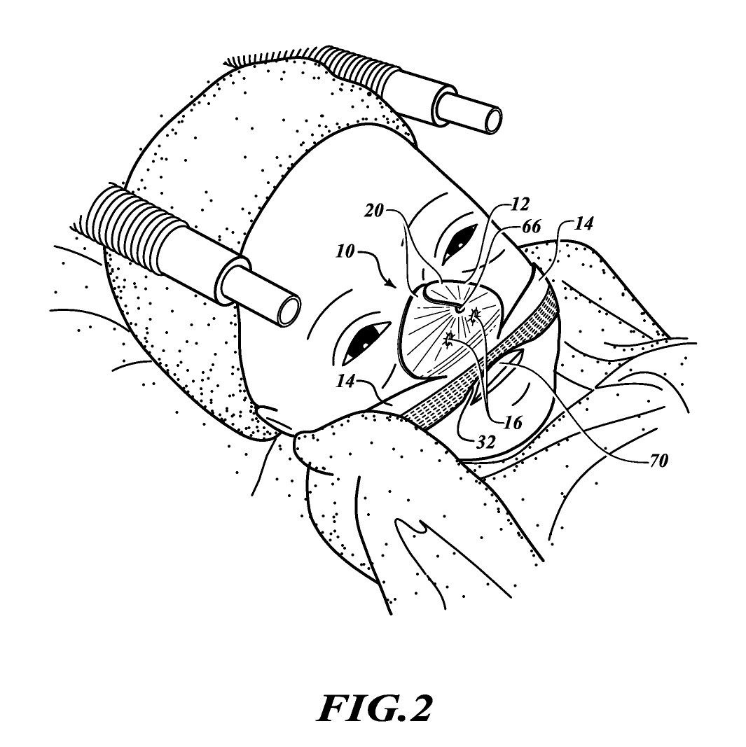

Lip and cheek portion 14 defines a cut-out 32, which helps medical personnel avoid placing a portion of the seal 10 on the vermillion 70 (FIGS. 2+3) of the upper lip. The vermillion is the red part of the lip, as opposed to the thicker-skinned portion that extends upwardly toward the nose and cheek. The vermillion 70 is very sensitive and ideally should not covered. Some have suggested that covering a portion of the vermillion 70 may interfere with an infants ability to begin nursing. Whether or not this is accurate, cutout 32 makes it easier to avoid placing a portion of seal 10 over the vermillion 70, to avoid any resulting problems, and to provide greater infant comfort.

As noted previously petals 20 define a notch 22. When applied about the infant nose, as shown in FIGS. 2 and 3, with one petal 20 folded onto another, this provides a vent 66 for condensation to escape. It had been noticed that the view of the nose afforded to medical personnel was sometimes obscured by cloudiness on the inner surfaces of petals 20. It is important that an unobscured view of the nose be available, so that medical personnel can monitor the condition of nose tissue. After analysis and experimentation it was determined that this cloudiness was caused by condensation that had no avenue of escape and built up as a layer on the inner surfaces of petals 20. This condensation also served to loosen the bond between the nose covering portion 12 and the underlying nose. By providing notch 22 and thereby vent 66 is permitted to escape and visibility of the nose is maintained. Also, the adhesive bond between nose covering portion 12 and nose is less vulnerable to attack by condensation.

Placement of the nostril apertures 16 is an exercise in delicate determination of a difficult to optimize location. To accommodate all nose shapes (representative of different racial groups and variations within racial groups) it has been found that optimally, even without the cut-out 32, it is important that apertures 16 be close to the bottom of lip and cheek adhering portion 14. Generally speaking, it is desirable that this distance be no greater than two center-to-center spacing lengths 24, and optimally less than one and one-half center-to-center spacing lengths 24.

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A nasal CPAP canula seal having directions of “above” and “below” by its anticipated placement on an infant face, above being higher up on said face and below being lower down, comprising:
(a) a canula supporting portion, defining holes for canula passage into the nostrils;
(b) a nose-anchoring portion extending about said apertures and designed to help anchor said nasal CPAP canula seal by adhering to the infant nose; and
(c) wherein said nose-anchoring portion defines a laterally central notch cut-out adapted to form a moisture vent when said nose-anchoring portion is folded about said nose.

2. The seal of claim 1, wherein said nose anchoring portion includes two petals, laterally abutting each other, and wherein said notch cut-out is located between said two petals.

3. The seal of claim 1, wherein said notch cut-out has about half the lateral width of a said aperture.

4. The seal of claim 1, made of a sheet of hydrocolloid laminated to polyurethane.

5. The seal of claim 1, defining a set of cuts emanating radially from said apertures.

6. A method of applying a nasal CPAP canula seal in such a manner that a vent is provided to permit moisture to escape from the nose skin, comprising:
(a) providing a nasal CPAP canula seal, including:
(i) a canula supporting portion, defining apertures for canula passage into the nostrils;
(ii) a nose-anchoring portion, attached to said canula supporting portion and defining a notch;
(b) placing said nasal CPAP canula seal about said infant nose so that said apertures are coincident with the infant's nostrils and said notch is substantially over the tip of the infant nose, thereby forming a vent that permits moisture to escape.

7. The method of claim 6, wherein said nose anchoring portion includes two petals, laterally abutting each other, and wherein said notch cut-out is located between said two petals.

8. The method of claim 6, wherein said notch cut-out has about half the lateral width of a said aperture.

9. The method of claim 6, wherein said canula seal is made of a sheet of hydrocolloid laminated to polyurethane.

10. The method of claim 6, wherein said canula seal define a set of cuts emanating radially from said apertures.

11. A nasal CPAP canula seal having directions of “above” and “below” by its anticipated placement on an infant face, above being higher up on said face and below being lower down and thereby defining latitude as a set of lines going horizontally across said canula seal, comprising:
(a) a canula supporting portion, defining two apertures for canula passage into the nostrils, said apertures defining a aperture-spacing length, said length being the distance between the centers of said two apertures;
(b) a nose-anchoring portion extending about said apertures and designed to help anchor said nasal CPAP canula seal by adhering to the infant nose;
(c) a lip and cheek adhering portion, defining the bottom of said seal; and
(d) wherein said aperture centers are within two aperture-spacing lengths of said bottom of said seal.

12. The seal of claim 11, wherein said aperture centers are within 1.5 aperture-spacing lengths of said bottom of said seal.

13. The seal of claim 11, made of a sheet of hydrocolloid laminated to polyurethane.

14. The seal of claim 11, further defining a set of cuts emanating radially from said apertures.

15. A nasal CPAP canula seal having directions of “above” and “below” by its anticipated placement on an infant face, above being higher up on said face and below being lower down, comprising:
(a) a canula supporting portion, defining holes for canula passage into the nostrils;
(b) a nose-anchoring portion extending about said apertures and designed to help anchor said nasal CPAP canula seal by adhering to the infant nose; and
(c) wherein said seal is made of material that has a skin-contacting layer having gentle adhesive qualities laminated to a shape-maintaining layer and wherein said material is transparent.

16. The seal of claim 15, wherein said skin-contacting layer is made of silicone adhesive.

17. The seal of claim 15, wherein said skin contacting layer may be washed without losing its adherent properties.

18. The seal of claim 15, wherein said skin contacting layer is made of biocompatible acrylic adhesive.

19. The seal of claim 15, wherein said shape maintaining layer is made of clear, biocompatible polymeric film.

20. The seal of claim 15, wherein said shape maintaining layer is made of silicone film.
